(19) United States
(12) Patent Application Publication
(10) Pub. No.: US 2006/0002886 A1
(43) Pub. Date: Jan. 5, 2006

(54) Waste water treatment biocatalyst - CIP

(76) Inventors: Henry B. Schur, Ft. Lauderdale, FL (US); Gary Morgan, Ft. Lauderdale, FL (US); Scott Windham, Ft. Lauderdale, FL (US)

Correspondence Address: Robert J. Van Der Wall;Gables One Tower, Suite #1275, 1320 South Dixie Highway, Coral Gables, FL, 33146, US

(21) Appl. No.: 11/217,714

(22) Filed: Sep. 2, 2005

Related U.S. Application Data
(63) Continuation-in-part of application No. 10/352,366, filed on Jan. 27, 2003.
(60) Provisional application No. 60/351,450, filed on Jan. 28, 2002.

(51) Int. Cl. A61L 9/00; A61L 9/01; A61K 35/78

(52) U.S. Cl. 424/76.1; 424/780

(57) ABSTRACT

Disclosed is a composition of matter that features a liquid biocatalyst to stimulate biological activity. The composition is both environmentally and physically safe. The biological simulators in the inventive biocatalyst increase the metabolic and reproductive rates of most bacteria. Its introduction into a waste water treatment system non-selectively enhances aerobic biological activity, thereby improving both carbonaceous and nitrogenous removals. The product is especially effective for endogenous situations. The non-selective nature of the product enhances most biological activity, thus allowing for overall performance improvements within a treatment plant. The inventive composition of matter is made by the blending of Ascophyllum nodosum seaweed extract, Liquid Coconut Oil Soap, Vitamin Mixture, and De-ionized water. Ascophyllum nodosum seaweed is preferably extracted from freshly harvested ascophyllum nodosum seaweed. The soap is a highly concentrated, versatile product made up of liquid coconut oil that is dispersing agent that acts as a lubricant or release agent. The vitamin mixture is used to supply the missing intra-cellular enzymes that are required for maximum cellular metabolism and utilization of the organic material in the waste water. These substances are very low or nonexistent in the normal waste water, industrial effluents and in most natural water environments.

### CROSS REFERENCE TO RELATED APPLICATIONS

The present Application is a Continuation In Part copending with an earlier application having the same title, filed Jan. 27, 2003, Ser. No. 10/352,366, which was copending with a previous provisional patent application filed Jan. 28, 2002, Ser. No. 60/351,450. The present Application claims the filing date thereof as to the common subject matter.

### BACKGROUND OF THE INVENTION

1. Field of the Invention

The present invention generally relates to the field of waste water treatment. Specifically, it concerns a new composition of matter in the form of a biocatalyst that stimulates metabolic and reproductive rates for most bacteria to greatly accelerate the process of waste water treatment.

2. Description of the Prior Art

The treatment of waste water in a conventional facility is a time consuming process. The result is that in order for any such facility to have meaningful capacity, the residence time of the waste water must be substantial in order for the bacteria to have sufficient time to achieve an acceptable effluent quality. This result in the construction of massive chemical storage tanks at great expense, which also constitute an eyesore in their communities.

Heinicke, U.S. Pat. No. 4,666,606, describes an extract of plant materials that produce a enzyme, xeronine, with the properties of bacteriological stimulation can be used in the waste water treatment field. This product is effective but has several drawbacks that the present invention overcomes. Mundschenk, U.S. Pat. No. 6,284.012, teaches a new method of extraction of the xeronine and a product derived there from the use in waste water treatment and grease removal in waste water lines. This product has the stated effect but has several shortcomings that the present invention improves upon and allows for a broader application.

### SUMMARY OF THE INVENTION

A principal object of the present invention is to greatly accelerate the process of waste water treatment.

A related object of the invention is to reduce the cost of future waste water treatment facilities by minimizing the need for massive chemical storage tanks to achieve sufficient residence time for the bacteria to achieve an acceptable effluent quality.

Another object of the invention is enhanced aerobic and anaerobic biological activity which in turn improves effluent quality.

A further object of the invention is decreased recovery time after upset in a treatment facility.

An additional object of the invention is bio-solids reduction via endogenous stimulation which with resulting lower solids disposal costs.

One more object of the invention is reduced scum formation resulting in less odors.

Another object of the invention is the elimination or reduction of algae.

An additional object of this invention is for the increase in metabolic rate of bacteria, plants, yeasts, and molds in the fermentation process, both as it relates to waste water and bio-solids digestion and other areas of microbial fermentation.

A further object of the invention is to increase yield of antibiotics and other biotechnology products.

An additional object of this invention is to increase the production of methane in an anaerobic digester to improve the yield of said production for co-generation of electric power or for conversion to methanol fuel.

A further object of this invention is to increase the biological metabolism of microorganisms present in animal wastes to reduce the formation of hydrogen sulfide gas and ammonia gas in waste lagoons and holding tanks.

An additional object of this invention is to reduce the sludge levels in waste lagoons and holding tanks thereby increasing their effective capacity.

Other objects and advantages will be apparent to those skilled in the art upon consideration of the following descriptions.

In accordance with a principal aspect of the invention, there is provided a liquid biocatalyst that stimulates biological activity. The product is both environmentally and physically safe. The biological simulators in the inventive biocatalyst increase the metabolic and reproductive rates of most bacteria. Its introduction into a waste water treatment system non-selectively enhances aerobic biological activity, thereby improving both carbonaceous and nitrogenous removals. The product is especially effective for endogenous situations. The non-selective nature of the product enhances most biological activity, thus allowing for overall performance improvements within a treatment plant.

The inventive composition of matter is made by the blending of Ascophyllum nodosum seaweed extract, Liquid Coconut Oil Soap (Concentrate 41), Vitamin Mixture, and De-ionized water. Ascophyllum nodosum seaweed is extracted from freshly harvested ascophyllum nodosum seaweed from the North Atlantic coast of Nova Scotia, Canada. The pH of the extract is 8. Concentrate 41 is a highly concentrated, versatile soap made up of liquid coconut oil that dispersing agent that acts as a lubricant or release agent. The pH of the concentrate is 9.9 and is odorless. The vitamin mixture is used to supply the missing intra-cellular enzymes and micro-nutrients that are required for maximum cellular metabolism and utilization of the organic material in the waste water. These substances are very low or nonexistent in the normal waste water, animal manure, industrial effluents and in most natural water environments.

The process of blending the concentrated product can be summarized as follows:

In a sanitized container add the calculated volume of plant extract then add with high shear mixing the surfactant (soap) until mixed; then add the required amount of prepared vitamin mixture again with high shear mixing at this time then add the preservative (methylparaben) and an anti-foam agent. The concentrate is diluted for use in a ratio of 1:11 (5 gal. To 55 gal) with the addition of sterile de-ionized water and additional preservative and anti-foam. The final product is adjusted to pH between 6.8 and 8.5 with citric acid solution.

### DETAILED DESCRIPTION OF THE PREFERRED EMBODIMENTS

As required, detailed embodiments of the present invention are disclosed herein; however, it is to be understood that the disclosed embodiments are merely exemplary of the invention which may be embodied in various forms. Therefore, specific functional details disclosed herein are not to be interpreted as limiting, but merely as a basis for the appended claims and as a representative basis for teaching one skilled in the art to variously employ the present invention in virtually any appropriate circumstance.

Preparation of the biocatalyst composition of matter is achieved with the following ingredients:
Extract: Ascophyllum Nodosum Liquid Seaweed Concentrate (29%) (Acadian Seaplants Limited, Nova Scotia, Canada)
Surfactant: Concord Soap Concentrate #41 (Concord Chemical Co., Camden, N.J.)
Vitamin mixture: (Florida Supplements Corp, Hollywood, Fla.)
Methyl paraben

De-ionized Water

| | | |
|---|---|---|
| B1 | 100 | mg/ml |
| Niacinamide | 100 | mg/ml |
| B6 | 10 | mg/ml |
| d-panthenol | 10 | mg/ml |
| B2 | 5 | mg/ml |
| B12 | 100 | mcg/ml |

Citric acid and benzyl alcohol 1.5% v/v as preservative
Preservative: (Nipa Chemical Co., UK)
Anti-Foam: FG-10 anti-foam (Dow Corning, Midland, Mich.)

The formula for one (1) gallon of the composition of matter at 6% solution is:

| Extract: | 227 | ml |
|---|---|---|
| Surfactant: | 94.6 | ml |
| Preservative: | 12.8 | ml |
| Vitamin: | 5.6 | ml |
| Anti-foam: | .05 | ml |
| Citric Acid: | Q.S. for pH adjustment | |
| DI Water: | 3428 | ml |

The preparation is described as follows:

To the required amount of DI water is added the seaplant extract and mixed with high shear mixing. The Surfactant is then added with mixing followed by the Vitamin mixture and the anti-foam. The Preservative is then added with high shear mixing. The entire batch is then pH adjusted with a 1N solution of citric acid to achieve a pH of between 6.8 and 8.5. The finished product is then dispensed into storage containers for use and stored at nominal room temperature. The product is stable for 1 year from the date of manufacture.

A sample of mixed liquor from the end of the aeration tank was collected to compare the characteristics of treated versus untreated sludge. A 30 minute Settleable Solids test, and a Total suspended solids test were run on the untreated sludge. The sample was then treated with 1.0 ppm of biocatalyst, and loaded into the respirometer. The respirometer continued to run for 24 hours until a constant endogenous rate of respiration was attained. The analyses were then repeated, in order to compare the results. The results are as follows:

| | |
|---|---|
| MLSS (Pre) = 3618 mg/l | MLSS (post) = 3065 mg/I |
| 30-min. Sett. (pre) = 43% | 30-min. Sett. (Post) - 35% |

The results indicate that the settling rate was improved by 18.6% and the suspended solids concentration was reduced by 15.3% following the addition of the biocatalyst. Observations were made of the results of the 30-min. Settleable Solids tests and some dramatic differences were noted. The supernatant liquid above the solid-liquid interface in the treated sample was clearer, with less turbidity than that of the untreated sample. The water surface appeared to be free of grease, oil and ash, where the untreated sample did not. Also, the sludge rose to the surface within a few hours.

Examples of laboratory records are as follows:

### Example 1

Oct. 31, 2001 On this day, an initial sampling was performed at the City of Sunrise, Florida Sawgrass water treatment plant, for the purpose of establishing laboratory procedures and to verify proper equipment operation. Samples were collected from the aeration basin, headworks and effluent and raw samples were analyzed for TSS. The results are as follows:
Mixed Liquor 30 min. Sett. Solids -56%
MLTSS - 3595 mg/l
Effluent TSS - 2.04 mg/l
Raw TSS - 128.8 mg/l
   The test results are as expected and the performance of all laboratory equipment is satisfactory.

### Example 2

Nov. 07, 2001 On this day, samples were collected at the Sawgrass facility, from the aeration basin and from the discharge manifold of the return activated sludge pumps (RAS). The purpose is to familiarize the technical staff in the operation of the respirometer and the interpretation of the respirometry graph results.

The respirometer was set up and calibrated and 1800 ml of mixed liquor from the aeration basin was added to the sample chamber. The instrument was run until a constant endogenous respiration rate was established. The rate was determined to be 7.4 ml/L/hr.

The sample chamber was drained, and 1800 ml of RAS was added. This sample was also run until a constant endogenous respiration rate was established. This rate was determined to be 9.0 ml/L/hr. The endogenous rate of the return activated sludge is typically three times higher than that of the mixed liquor. It was determined that a process modification was made by the plant operator, where raw wastewater was entering the aeration basin, immediately upstream of the clarifier. This would account for the anomalous rate of respiration. All subsequent samples of aeration basin mixed liquor shall be collected from the northern basin of the “new side” of the facility, immediately prior to the clarifiers. In the future, Return Activated Sludge (RAS) samples will not be collected from this facility.

### Example 3

Nov. 08, 2001 On this day, the RAS sample from the previous analysis was retained in the sample chamber for another series of teats, The endogenous rate of respiration was 11.50 ml/L/hr at the start of the test procedure was to add increasing amounts of food (beer), and determine the initial respiration rates, and time required to metabolize the food (treatment time). The results of the tests are as follows:
3 ml:
   IRR=26.17 ml/l/HR
   TT=102 min.
6 ml:
   IRR=44.56 ml/L/hr
   TT=123.6 min.
9 ml:
   IRR=48.12 ml/l/HR
   TT=140.4 MIN.

The test results demonstrate the increasing respiration rates and treatment times due to the respective amounts of added food.

### Example 4

(3 ml Beer)
   RR-71.15 ml/L/hr
   TT=24 min.
(3 ml Beer+2 ppm) RR=81.4 ml/L/hr
% Increase RR (w/ the biocatalyst) =12.2%
% Decrease TT (w/ the biocatalyst) =12.6%

### Example 5

Nov. 26, 2001 On this day, a sample of mixed liquor (2000 ml) from the end of the aeration basin was collected and loaded into the respirometer. The sample continued to run until a constant endogenous rate of respiration was attained. This rate was determined to be 9.65 ml/L/hr. 3 ml of beer was added to the sample and the respiration rate was recorded. The respiration rate was 24.75 ml/L/hr and the treatment time was 109 minutes. The sample was then treated with 3 ml of beer+2 ppm of the biocatalyst. The respiration rate was 30.77 ml/L/hr and the treatment time was 81.6 minutes. The respiration rate was increased 19.6% and the treatment time reduced 25.1%.

The test was continued with the addition of 6 ml of beer to the sample. The respiration rate was 38.6 ml/L/hr and the treatment time was 112.8 minutes. The sample was then treated with 6 ml of beer +2 ppm of the biocatalyst. The respiration rate was 47.0 ml/L/hr and the treatment time reduced 18.1%.

### Example 6

Nov. 28, 2001 On this day, a fresh sample of mixed liquor (2000 ml) from the end of the aeration basin was collected and located into the respirometer. The sample continued to run until a constant endogenous rate of respiration was attained. This rate was determined to be 8.69 ml/L/hr. The sample was treated on an alternating basis with 3 ml of beer, then 3 ml of beer +2 ppm of the biocatalyst. A total of 4 series of test were run on this basis.

The results of the tests are as follows:

| | | |
|---|---|---|
| Test #1 | (3 ml Beer) | RR = 27.47 ml/L/hr |
| | | TT = 102 min. |
| | (3 ml Beer + 2 ppm) | RR = 30.77 |
| | | TT = 87.6 min. |

% Increase RR (w/the biocatalyst) = 10.7%
% Decrease TT (w/the biocatalyst) = 14.1%

| | | |
|---|---|---|
| Test #2 | (3 ml Beer) | RR = 36.05 ml/L/hr |
| | | TT = 62.4 min. |
| | (3 ml Beer + 2 ppm) | RR = 41.65 ml/L/hr |
| | | TT = 52.2 min. |

% Increase RR (w/the biocatalyst) = 13.4%
% Decrease TT (w/the biocatalyst) = 16.3%

| | | |
|---|---|---|
| Test #3 | (3 ml Beer) | RR = 56.71 ml/L/hr |
| | | TT = 34.8 min. |
| | (3 ml Beer + 2 ppm) | RR = 66.91 ml/L/hr |
| | | TT = 30 min. |

% Increase RR (w/the biocatalyst) = 15.2%
% Decrease TT (w/the biocatalyst) = 13.8%

### Example 7

Example of Product over raw extract on test organisms:

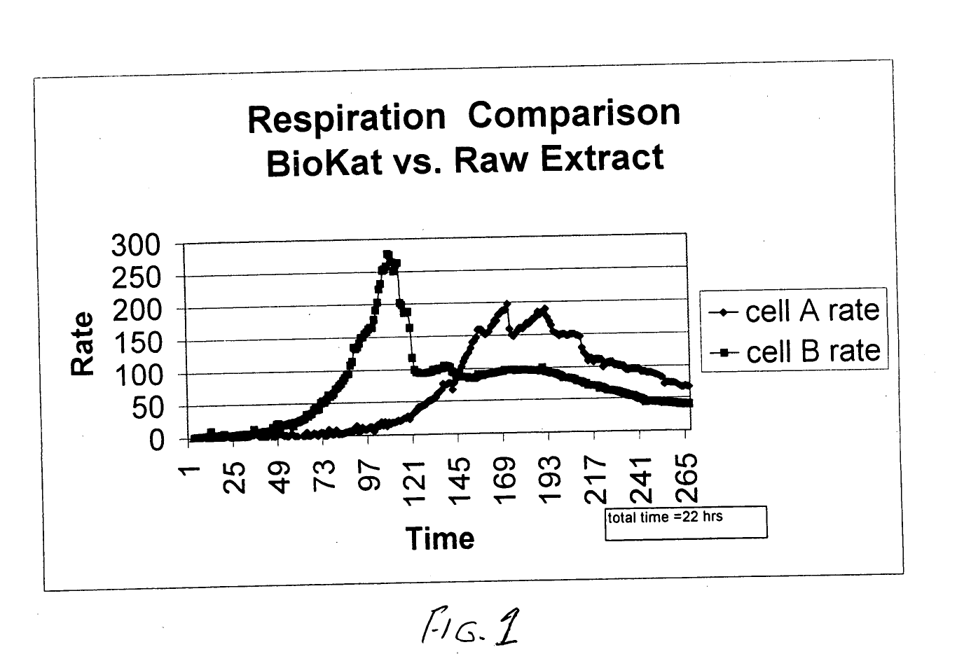

This test was a respiration comparison, and the date is shown on FIG. 1. It was performed in an Arthur Technologies Duel Chamber Respirometer at 25° C. Cell A contained a standard seed culture of microorganisms and nutrient common to the waste water industry and the addition of the raw sea plant extract. Cell B contained the exact same mixture of organisms and nutrients but this cell had the formula as presented herein in place of the raw extract. The resultant data shows the marked increase in cellular respiration with the formula vs. the raw extract and the log phase growth of the organisms occurs many hours before the log phase growth in the cell with only the extract.

### Example 8

Example of Product in a working sewerage collection system:

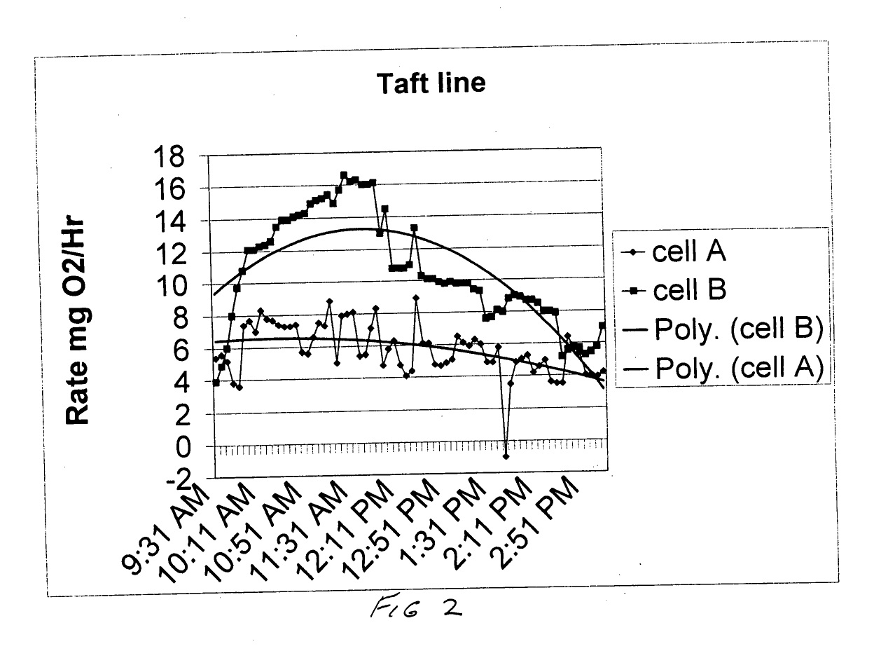

Reference is made to FIG. 2. This Taft Line chart is of a controlled study performed on an existing main sewerage collection point in a municipality in South Florida. The sample was taken from a manhole in a sterile container and returned to the lab within 2 hours. The sample was placed in the Respirometer so that cell A contained the raw sewage without additives and cell B contained the same amount of material plus the addition of the formula of this invention. It is very apparent that the results of respiration (oxygen utilization per hour) are substantially increased by the use of this invention. This test confirms that the addition of this inventive product does increase the respiration of the microorganisms present which results in an increase utilization of the nutrients present in waste water. This increased utilization results in a decrease in biochemical oxygen demand (BOD) and a reduction in sludge volume.

### Example 9

Example of the product to reduce the fats, oil and grease (FOG) in a pump/lift station:

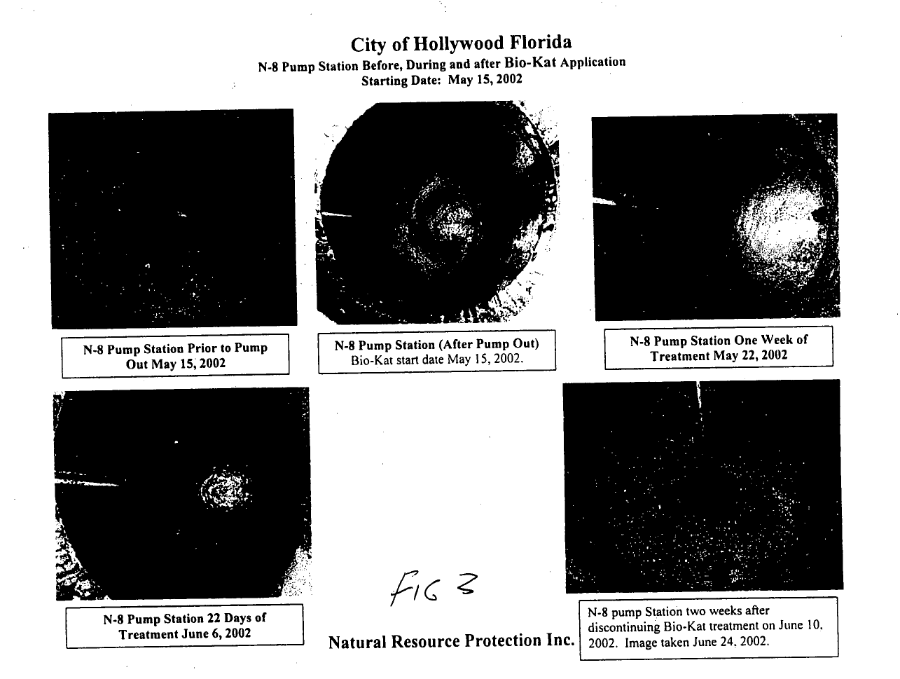

Reference is made to FIG. 3, a series of color photographs necessary to show the efficacy of the invention and believed to qualify for inclusion in the application by reason of 37 C.F.R. § 1.84 (b) (2). This is a photographic documentation of the N-8 pump station in a municipal system in South Florida where the accumulation of FOG required the station to be pumped out (cleaned) about every two weeks. This was a very expensive and time consuming process and thus a solution was sought to remedy it. We introduced the present invention through a programmed dispenser above the surface of the nominal flow level in the “pit”. After approximately one month o use the station remained clear of any FOG build up and did not require any pump out. As the photos show, after the discontinuation of the product, the FOG problem returned within two weeks and required pumping to clean it out. The product has since been put into continuous use with in this municipality where it continues to perform as indicated.

### Example 10

A hog farm (Morris Farms) in western Kansas was selected for a detailed scientific study of the product in both the control of odor and lagoon chemistry. The results are detailed in the paper attached. The results showed a significant reduction in ammonia within the hog barns and a marked improvement in the lagoon chemistry.

### Example 11

This study was undertaken at the Philadelphia biosolids treatment facility in Philadelphia, Pa. to examine the effect of the product on the production of methane and the reduction of mercaptans (odor producing chemicals) in the sludge storage and digester tanks. The data clearly shows a several fold increase in the methane production with a drop of mercaptans to near zero. The report is attached.

While the invention has been described, and disclosed in various terms or certain embodiments or modifications which it has assumed in practice, the scope of the invention is not intended to be, nor should it be deemed to be, limited thereby and such other modifications or embodiments as may be suggested by the teachings herein are particularly reserved especially as they fall within the breadth and scope of the appended claims.

What is claimed is:

1. A composition of matter having a mechanism of cellular metabolic increase to optimize efficiency in processes that depend upon living organism metabolism for their operation and/or product production.

2. The composition of claim 1 used to treat all waste water to reduce its overall biochemical oxygen demand, solids content and improve its settability.

3. The composition of claim 1 used to increase production of extracellular products by stimulation of beneficial microorganisms to increase their utilization substrates.

4. The composition of claim 1 used to increase production of alcohol and other fermentation processes using microorganisms.

5. The composition of claim 1 used to increase yield of antibiotics and other biotechnology products produced from cultivation of microorganisms where the composition is used to activate the microorganisms to a greater rate of respiration and thus conversion of substrate to usable product.

6. The composition of claim 1 that is used to reduce hydrogen sulfide odor from a waste water system.

7. The composition of claim 1 used to optimize agricultural supplements including fertilizers and crop stimulants by stimulation of soil microorganisms.

8. The composition of claim 1 used to control aquatic algae growth in ponds, lakes and lagoons by stimulation of microorganisms and their consumption of otherwise available nutrients.

9. The composition of claim 1 added to commercially used microorganisms to increase their effectiveness for their designated purpose.

10. The composition of claim 1 used to optimize environmental remediation of hydrocarbon spills.

11. The composition of claim 1 used to increase the production of methane in anaerobic environments.

12. The composition of claim 1 used to reduce the TKN in wastewater.

13. The composition of claim 1 used to reduce the sludge in lagoons, digesters and sludge storage tanks.

14. The composition of claim 1 used to reduce odors in animal housing units and CAFO's.
